(19) United States
(12) Patent Application Publication
(10) Pub. No.: US 2013/0252254 A1
(43) Pub. Date: Sep. 26, 2013

(54) Procalcitonin gene expression as a precise biomarker of aging process

(71) Applicant: Mehmet Ali Soylemez, Istanbul (TR)

(72) Inventor: Mehmet Ali Soylemez, Istanbul (TR)

(73) Assignee: Mehmet Ali Soylemez, Istanbul (TR)

(21) Appl. No.: 13/506,278

(22) Filed: Apr. 9, 2012

Related U.S. Application Data
(60) Provisional application No. 61/613,708, filed on Mar. 21, 2012.

(51) Int. Cl. G01N 33/566; G01N 21/64; C12M 1/34; G01N 33/573

(52) U.S. Cl. 435/7.4; 436/501; 435/287.2; 422/69

(57) ABSTRACT

The invention relates to a method for diagnosis and/or complications and/or risk assessment of aging, in particular of aging process, wherein a determination of the marker procalcitonin (PCT: SEQ ID No. 1) or a partial peptide or fragment thereof or if contained in a marker combination (Panel, Cluster) is carried out on a patient within normal value ranges of leucocytes under investigation. The invention further relates to invent new drugs, a diagnostic device and a kit for carrying out said method.

The Invention relates to a method for the diagnosis and/or complications and/or risk stratification of aging, particularly of aging process, wherein a determination of the marker procalcitonin (PCT: SEQ ID No. 1) or a partial peptide or fragment thereof, or contained in a marker combination (panel, cluster), is carried out on a patient within normal value ranges of leucocytes to be investigated. Furthermore, the invention relates to a diagnostic device and a kit for carrying out the method. The diagnostic device and a kit must be used only on a patient within normal value ranges of leucocytes. Otherwise, the diagnostic device and the kit should not be used for diagnostic test of aging process and/or complications.

The diagnosis of aging process and/or complications is not known for procalcitonin until our on the bases of procalcitonin genetic expression experimental design and study Relationship between plasma procalcitonin levels and aging process in patients. However, other aging follow up markers are disadvantageous, because that the lack of a human body injury marker of aging process, results in no reliable diagnosis can take place. Therefore, there is a need for presenting on the bases of genetic mechanism and a reliable diagnosis and risk assessment of aging process, or for undertaking a (risk) stratification, particularly with regard to further clinical decisions and, in particular, with regard to the degree of severity of aging or aging sequelae. It has been claimed first time in this patent application, that this genetic mechanism works only for patients within normal value ranges of leucocytes for diagnosis and/or complication risk assessment of aging process. Otherwise, the results of test will not show the diagnosis and/or complication risk assessment of aging.

Furthermore, the procalcitonin (PCT) determination in diagnosis is described in the state of the art, particularly with regard to an Investigation of sepsis (Yukioka et al., Ann. Acad. Med. Singapore 30: 528-31 (2001). However, the suitability of procalcitonin (PCT: SEQ ID No. 1) for the diagnosis and complications of aging process are not disclosed.

It is the task of the present invention to make available an improved method for the diagnosis and/or aging complications and/or risk stratification of aging process, particularly of aging sequelae.

This task is accomplished by means of a method for in vitro diagnosis and/or risk stratification of aging, wherein a determination of the marker procalcitonin (PCT: SEQ ID No. 1) or a partial peptide or fragment thereof, or contained in a marker combination (panel, cluster), is carried out in a patient within normal value ranges of leucocytes to be investigated (referred to hereinafter as method according to the invention).

The term “risk stratification,” according to the invention, comprises finding human aging patients, particularly those having aging sequelae, with the worse prognosis, for the purpose of intensive diagnosis and therapy/treatment (of sequelae) of aging process, with the goal of allowing as advantageous a course of the aging process as possible.

For this reason, it is particularly advantageous that a reliable diagnosis and/or risk stratification can take place by means of the method according to the invention. The method according to the invention allows clinical decisions that lead to a more rapid diagnosis, particularly of the aging sequelae and prognosis. Such clinical decisions also comprise further treatment using medications, for the treatment or therapy of aging process and complications.

Invention is related to possible a new drug development (anti-aging drugs) for the treatment of aging.

In serum, procalcitonin (PCT) has a half-life of 25 to 30 hours (Karzai et al. Procalcitonin—A New Indicator of the Systemic Response to Severe Infections. INFECTION Volume 25, Number 6, 329-334) Therefore, to the invention, a daily rapid or normal PCT test result could give enough knowledge for evaluation of aging and/or aging damage in 1-day period. So, it is possible to prevent daily aging and/or aging complication and/or complications damage by determining the right drug and diet therapy and/or invent new drugs and explore of new diet therapies for aging process.

In vitro method for prognosis for a patient having aging, the method comprising: determining the level of procalcitonin or fragments in a sample within normal value ranges of leucocytes selected from the group consisting of a saliva, a blood sample, a serum sample and a plasma samples obtained from the patient; and correlating the level of procalcitonin or fragments thereof to the risk of the patient to contract a further disease or medical condition which has been manifested or not yet manifested and/or is symptomatic or not yet symptomatic, wherein the correlating step comprises comparing the level of procalcitonin or fragments thereof to a threshold level, whereby, when the level of procalcitonin or fragments thereof exceeds the threshold level, the patient is predisposed to the risk of aging and/or aging complications, and wherein the threshold level is 0.03±0.002 ng/mL (Please see: Table 2, Table-1).

Normal value ranges of procalcitonin threshold level at aging process diagnosis and/or complications and/or risk strafication may vary slightly among different laboratories.

In another preferred embodiment of the method according to the invention, diagnosis and/or risk stratification take place for prognosis, for prophylaxis, for early detection and detection by means of differential diagnosis, for assessment of the degree of severity, and for assessment of the course of aging process and complications as an accompaniment to the therapy.

In another preferred embodiment of the method according to the invention, samples of body fluids, saliva, particularly blood, optionally whole blood, serum, or available plasma, are taken from the patient to be investigated, and the diagnosis takes place in vitro/ex vivo, i.e. outside of the human or animal body. The diagnosis and/or risk stratification can take place on the basis of the determination of the marker procalcitonin (PCT: SEQ ID No. 1) or partial peptides or fragments thereof, and its amount that is present, or a change in amount, as compared with a reference, in at least one patient sample.

Within the scope of this invention, the term “aging,” in humans refers to a multidimensional process of physical, psychological and social change. Some dimensions of ageing (aging) grow and expand over time, while others decline. Roughly 100.000 people worldwide die each day of age related diseases. Age is measured chronologically. However the term ‘ageing’ somewhat ambiguous. Distinctions may be made between universal aging (age changes that all people share) and probabilistic aging (age changes that may happen to some, but not all people as they grow older including diseases such as type 2 diabetes mellitus). Inflammation is the single greatest precipitator of aging and age related diseases such as diabetes mellitus, heart disease, alzheimer, arthritis, certain forms of cancer, as well as wrinkled sagging skin. Inflammation which takes place on a cellular level is triggered by a wide variety of factors such as oxidative stress, ingestion of toxins, excess exposure to ultraviolet radiation, hormonal changes and eating a pro-inflammatory diet (hyperglycemic diet). Procalcitonin is elevated by inflammatory processes, with greater specificity and sensitivity than other acute phase proteins (Hatheril M., Tibby S M., Sykes K., Turner C., Murdoch, I A., Diagnostic markers of infection: comparison of procalcitonin with C reactive protein and leucocyte count. Arch Dis Child). For this reason, the invention relates to the diagnosis and/or risk stratification of aging process, and its sequelae and concomitant illnesses, particularly endothelial dysfunction, hyperlipoproteinemia, hypertensive dysregülation, cardiovascular complications and diabetes mellitus etc. . . .

Within the scope of this invention, “Procalcitonin (abbreviated as: PCT) was originally described in 1984 as a 116-aminoacid protein with a molecular weight of 14.5 kDa (Le Moullec J. M., et al. The complete sequence of human procalcitonin. FEBS Lett., 167 (1), 93-97 (1984)). The PCT gene, referred to as Calc-1, is located on chromosome 11p15.4 and was sequenced in 1989. The promoter has sites for basal transcription factors but more interestingly, also has sites for NF .kappa..beta. (Nuclear factor .kappa..beta.) and AP-1(Activator protein-1), factors induced under inflammatory conditions (Whicher J., Bienvenu J., Monneret G., Procalcitonin as an acute phase marker. Ann Clin Biochem. 38 (5):483-93, 2001). Hyperglycemia is associated with oxidative stress and elevation of advanced glycation end products (AGEs). AGEs are produced by a non-enzymatic, Maillard reaction between reducing sugars and either proteins or lipids. AGEs interact the receptor for advanced glycation end products (RAGE) and, RAGE activation is caused by elevation of transcriptional factors NF .kappa..beta. and AP-1 (Beckman J., Creager M., Libby P., Diabetes and Atherosclerosis JAMA 287: 2570-2581, 2002). These factors (NF .kappa..beta. and AP-1) induce procalcitonin gene expression (Mehmet Ali Soylemez et al. A Novel Mechanism between Type II Diabetes Mellitus and Procalcitonin Gene Expression Molecular Therapy (2005) 11, S3461[ndash]1S346; doi: 10.1016/j.ymthe.2005.07.437). This algorithmic mechanism was based on genetic expression of procalcitonin and first time showed by our study. Results of our study and experimental design imply procalcitonin as an important mediator during the development of aging process and/or diabetes mellitus. Thus, the results of this study show that procalcitonin (PCT: SEQ ID No. 1) is a new marker for aging process and diabetes mellitus and/or diabetic patients with complications (Mehmet Ali Soylemez; ‘Procalcitonin is a specific marker of diabetic complication process’(ESHG conference 2010 Gothenburg, Sweden; EJHG volume 18 supl. 1, Metabolic disorders Page: 352, P 13.14).

In another embodiment, the determination of procalcitonin (PCT: SEQ ID No. 1) can additionally take place with other markers, where the procalcitonin (PCT: SEQ ID No. 1) is contained in a marker combination (panel, cluster), specifically and preferably with those that already indicate aging. In another particular embodiment, this can also be a vascular marker that can indicate an endothelial dysfunction of the cardiovascular system that accompanies aging.

For this reason, the Invention relates to an embodiment of the method according to the invention where the determination is additionally carried out with at least one further marker selected from the group of inflammatory markers, vascular markers, in a patient to be investigated.

According to the invention, the inflammatory marker can be selected from at least one marker of the group of C-reactive protein (CRP), cytokines, such as TNF-alpha, for example, interleukins, such as IL-6, interleukin-1.beta. Proadrenomedullin (proADM), midregional proadrenomedullin (MR-proADM) angiotensin II, endothelin-1, and adhesion molecules, such as VCAM or ICAM, and the vascular marker can be selected from at least one marker of the group of creatine kinase, myeloperoxidase, myoglobin, natriuretic protein, particularly ANP (or ANF), proANP, NT-proANP, BNP, proBNP, NT-proBNP, or a partial sequence thereof, in each instance, CRP. Furthermore, this term is also understood to mean (pro)hormones that regulate the cardiovascular system, particularly such as pro-gastrin-releasing peptide (proGRP), pro-endothelin-1, pro-leptin, pro-neuropeptide-Y, pro-somatostatin, pro-neuropeptide-YY, pro-opiomelanocortin, or a partial sequence thereof, in each instance.

In another embodiment, at least one diabetic and aging marker/factor can additionally be determined. Diabetic and aging markers/factors are, according to the invention, particularly those such as adiponectin, carbohydrates, fats, such as cholesterols (LDH) and others, Body Mass Index (BMI), age, blood pressure, HOMA-IR (Homeostasis Model Assessment-insulin Resistance index, for a determination see: Matthews D R, Hosker J P, Rudenski A S, Naylor B A, Treacher D F, Turner R C, Homeostasis Model Assessment: Insulin Resistance and B-cell Function from Fasting Plasma Glucose and Insulin Concentrations in Man. Diabetologia 28:412-419. 1985).

In another embodiment of the Invention, the method according to the invention can be carried out by means of parallel or simultaneous determinations of the markers (e.g. multi-titer plates with 96 cavities and more); where the determinations are carried out on at least one patient sample.

Furthermore, the method according to the invention and its determinations can be carried out using an automated analysis device, particularly using a Kryptor (http://www.kryptor.net/).

In another embodiment, the method according to the Invention and its determinations can be carried out by means of a rapid test (e.g. lateral flow test), whether using single-parameter or multi-parameter determinations.

Furthermore, the Invention relates to the use of procalcitonin (PCT: SEQ ID No. 1) or partial peptides or fragments thereof, or contained in a marker combination (panel, cluster), for in vitro diagnosis and/or complications and/or risk stratification of aging, particularly aging process and its sequelae and concomitant illnesses, as well as, in particular, taking the aforementioned embodiments into consideration. The marker combination can contain another suitable marker, if necessary.

Another task is making available a corresponding diagnostic device, or the use of such a device for carrying out the methods according to the Invention.

Within the scope of this invention, such a diagnostic device is particularly understood to be an array or assay (e.g. immune assay, ELISA, etc.), in the broadest sense a device for carrying out the method according to the invention.

The invention furthermore relates to a kit or the use of such a kit for in vitro diagnosis or risk stratification of ageing, particularly ageing process, and its sequelae and concomitant illnesses, where a determination of procalcitonin (PCT: SEQ ID No. 1) or partial peptides or fragments thereof, or contained in a marker combination (panel, cluster), is carried out in a patient to be investigated, particularly taking into consideration the aforementioned embodiments. Such detection reagents comprise antibodies, etc. . . .

The following examples and tables serve for a more detailed explanation of the invention, but without restricting the invention to these examples and tables.

### EXAMPLES

### Example 1

Serum procalcitonin levels were analyzed with kryptor analyzator. Kryptor-PCT is a kit designed for kryptor automated immunofluorescent assay of procalcitonin in serum.

Statistical analysis was performed by using SPSS method. The results were expressed as mean.+−.standard deviation. Individuals between group comparisons were made using Student's-T test. Significance was defined at P<0.05

All test subjects of blood leucocytes count was normal and within normal value ranges of leucocytes (Our Laboratory normal value ranges of leucocytes: 4000/mm3-10000/mm3).

Our laboratory normal value ranges of blood sugar: 80-110 mg/dl

Procalcitonin was determined in 14 healthy, normo-glycemic test subjects with undisturbed glucose tolerance within normal value ranges of leucocytes, 16 patients having manifest hyperglisemic-diabetes mellitus type 2 without complications and within normal value ranges of leucocytes (abbreviated as: “DM II”).

Table-2 shows procalcitonin in healthy test subjects with normo-glycemia, and patients having diabetes mellitus (DM II) with hyperglycemia (Table-1).

Table-1 and 2 shows not only Procalcitonin but also the parameters relevant to hyperglycemia, such as glucose, HbAlc and leucocyte in the groups, in each instance.

Procalcitonin (PCT) levels were elevated in patients within hyperglycemia when compared with normal, normo-glycemic subjects. There was a statistically significant difference in serum procalcitonin of type 2 diabetic patients within hyperglycemia versus healthy normo-glycemic subjects (P<0.01) (see Table. 3).

### Tables

Table 1 shows not only procalcitonin but also the parameters relevant to hyperglycemia, such as blood sugar (glucose), HbAlc and leucocyte count, in the patient groups with hyperglycemia, in each instance.

Table 2 shows not only procalcitonin but also glucose, HbAlc and leucocyte values, in the control (non-hyperglycemic) groups, in each instance.

Table 3 shows PCT statistical differences of between patient group with hyperglycemia and control (non-hyperglycemic) groups.

TABLE 1
Table-1: Hyperglycemic patient group parameters: blood sugar,
procalcitonin, HbA_{1c} and leucocyte values.

| | Blood Sugar | Procalcitonin | HbA_{1c} | Leucocyte |
|---|---|---|---|---|
| Patient No: | (mg/dl) | (ng/ml) | (%) | (/mm³) |
| D-1 | 179 | 0.0489 | 8.1 | 6440 |
| D-2 | 248 | 0.0190 | 10.1 | 8480 |
| D-3 | 223 | 0.0291 | 9.4 | 5370 |
| D-4 | 123 | 0.0392 | 8.5 | 7890 |
| D-5 | 157 | 0.0190 | 7.0 | 7920 |
| D-6 | 158 | 0.0367 | 10.4 | 6330 |
| D-7 | 172 | 0.0553 | 7.5 | 9140 |
| D-8 | 304 | 0.0333 | 11.2 | 7770 |
| D-9 | 138 | 0.0190 | 7.2 | 5130 |
| D-10 | 370 | 0.0190 | 10.4 | 6440 |
| D-11 | 168 | 0.0710 | 8.0 | 8070 |
| D-12 | 155 | 0.0252 | 9.7 | 8880 |
| D-13 | 355 | 0.0409 | 11.6 | 5770 |
| D-14 | 170 | 0.0190 | 9.2 | 7700 |
| D-15 | 289 | 0.0650 | 9.3 | 8560 |
| D-16 | 144 | 0.0740 | 5.9 | 6800 |
| Arithmetic | 209.56 | 0.03835 | 8.97 | 7293.1 |
| average: | | | | |
| SD: | 79.46 | 0.0193 | 1.605 | 1265.2 |

TABLE 2
Table-2: Control group parameters: blood sugar, procalcitonin, HbA_{1c} and
leucocyte values.

| | Blood Sugar | Procalcitonin | HbA_{1c} | Leucocyte |
|---|---|---|---|---|
| Control No | (mg/dl) | (ng/ml) | (%) | (/mm³) |
| C-1 | 101 | 0.0224 | 6.1 | 5120 |
| C-2 | 102 | 0.0190 | 5.5 | 5880 |
| C-3 | 102 | 0.0269 | 6.2 | 6330 |
| C-4 | 89 | 0.0220 | 5.6 | 4740 |
| C-5 | 92 | 0.0190 | 5.8 | 5420 |
| C-6 | 96 | 0.0281 | 5.4 | 6270 |
| C-7 | 93 | 0.02 | 5.8 | 5970 |
| C-8 | 90 | 0.024 | 5.6 | 7890 |
| C-9 | 94 | 0.023 | 5.8 | 5220 |
| C-10 | 93 | 0.022 | 5.7 | 7300 |
| C-11 | 90 | 0.0190 | 5.7 | 7270 |
| C-12 | 91 | 0.0190 | 5.6 | 6300 |
| C-13 | 97 | 0.0260 | 5.6 | 6390 |
| C-14 | 80 | 0.028 | 6.1 | 5730 |
| Arithmetic | 93.57 | 0.0227 | 5.75 | 6130.7 |
| Average: | | | | |
| SD: | 5.92 | 0.0034 | 0.237 | 896.78 |

TABLE 3
Table-3: PCT statistical differences of patient and control groups:

| | Control Group: | Patient Group: |
|---|---|---|
| | (Avg ± S.D) | (Avg ± S.D) |
| PCT | 0.0227 ± 0.0034 | 0.03835 ± 0.0193** |
| (ng/ml) | | |

**p < 0.01

Sequence listing shows the results of procalcitonin (abbreviated as: “PCT”) with the related partial sequences.

1. Method for in vitro diagnosis and/or complications and/or risk stratification of aging process, comprising determining procalcitonin (PCT: SEQ ID No. 1) or partial peptides or fragments thereof in a patient within normal value ranges of leucoytes to be investigated.

2. Method according to claim 1, characterized in that in vitro diagnosis and/or risk stratification of aging process and its sequelae and concomitant illnesses and cardiovascular complications take place.

3. Method according to claim 1, further comprising determining at least one further marker selected from the group of inflammatory markers, vascular markers, and/or aging markers/factors in a patient to be investigated.

4. Method according to claim 3, characterized in that the inflammatory marker is selected from at least one marker of the group of C-reactive protein (CRP), cytokines, such as TNF-alpha, for example, interleukins, such as IL-6, interleukin-1.beta, Proadrenomedullin (proADM), midregional proadrenomedullin (MR-proADM), angiotensin II, endothelin-1.

5. Method according to claim 3, characterized in that the vascular marker is selected from at least one marker of the group of creatine kinase, myeloperoxidase, myoglobin, natriuretic protein, particularly ANP (or ANF), proANP, NT-proANP, BNP, proBNP, NT-proBNP, or (pro)hormones that regulate the cardiovascular system, such as pro-gastrin-releasing peptide (proGRP), pro-endothelin-1, pro-leptin, pro-neuropeptide-Y, pro-somatostatin, pro-neuropeptide-YY, pro-opiomelanocortin, or a partial sequence thereof, in each instance.

6. Method according to claim 3, characterized in that parallel or simultaneous determinations of the markers are carried out.

7. Method according to claim 1, characterized in that the determinations are carried out on at least one patient sample.

8. Method according to claim 1, characterized in that the determinations are carried out using an automated analysis device.

9. Method according to claim 1, characterized in that the determinations are carried out by means of a rapid test.

10. Method according to claim 1, characterized in that the diagnosis is for the stratification of patients for clinical decisions related to treatment or therapy of aging and/or complications.

11. Method according to claim 1, characterized in that the diagnosis and/or risk stratification takes place for prognosis, for prophylaxis, for early detection and detection by means of differential diagnosis, for assessment of the degree of severity, and for assessing the course of aging, particularly aging process, and its concomitant illnesses and sequelae, as an accompaniment to therapy.

12. Method according to claims 1,2 and 11 invention is related to the development of new drugs and therapy procedures of anti-aging and longevity.

13. Diagnostic device for carrying out a method according to claim 1.

14. Kit for in vitro diagnosis and/or risk stratification of aging, particularly aging process, and its concomitant illnesses and sequelae, containing detection reagents for determining the marker procalcitonin (PCT: SEQ ID No. 1) or partial peptides or fragments thereof, or contained in a marker combination, wherein the marker combination contains other markers according to claim 3, and ancillary substances.


SEQUENCE LISTING (1 sequence)

SEQ ID NO: 1 - protein, 116 aa, Homo sapiens
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN